(19) United States
(12) Patent Application Publication
(10) Pub. No.: US 2006/0235279 A1
(43) Pub. Date: Oct. 19, 2006

(54) Less invasive access port system and method for using the same

(76) Inventors: David T. Hawkes, Pleasant Grove, UT (US); Thomas M. Sweeney II, Sarasota, FL (US); Michael D. Ensign, Salt Lake City, UT (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/384,139

(22) Filed: Mar. 17, 2006

Related U.S. Application Data
(60) Provisional application No. 60/663,094, filed on Mar. 18, 2005.
(60) Provisional application No. 60/685,185, filed on May 26, 2005.
(60) Provisional application No. 60/703,606, filed on Jul. 29, 2005.

(51) Int. Cl. A61B 1/32

(52) U.S. Cl. 600/222

(57) ABSTRACT

A less invasive access port for use in minimally invasive surgery allows for manipulation of the viewing angle into the working site in a transverse plane. According to one exemplary embodiment, the less invasive access port is designed to minimize the need for muscle retraction. Additionally, the less invasive access portal provides sufficient light, irrigation, suction and space for sundry medical instruments. According to one exemplary embodiment, a less invasive access port device includes a two-piece retractor having locking arms secured by a latch. The latch is located outside a wound during operation for ease of access. A cannula includes integrated interfaces for light, irrigation and suction. A housing forms a collar around a top of the cannula and houses the light, irrigation and suction mechanisms. Instruments and implants may be passed through the cannula and into the working space created by the two-piece retractor. Visualization of the working site can be attained under direct vision.

### RELATED APPLICATIONS

This application claims the benefit under 35 U.S.C. §119(e) of U.S. Provisional Patent Application No. 60/663,094 filed Mar. 18, 2005 and U.S. Provisional Patent Application No. 60/685,185 filed May 26, 2005, both of which are titled “Less Invasive Access Port,” as well as U.S. Provisional Patent Application No. 60/703,606 filed Jul. 29, 2005 titled “Minimally Invasive Surgical Retractor.” The provisional applications are incorporated herein by reference in their entireties.

### FIELD

The present system and method relate to devices and methods for performing percutaneous surgeries, and more particularly, to a less invasive access portal for use in orthopedic spinal surgery.

### BACKGROUND

Traditionally, the surgical exposure employed to perform spinal surgery inflicts significant and long lasting damage to the surrounding soft tissues. Surgical exposure, commonly referred to as an ‘open’ procedure, relies on retraction of muscles to open a channel to the underlying bony structures. Surgical retractors are often used to provide the operating channel. Common surgical retractors as used in the art today include rakes, forks, and different sized and shaped hooks. Normally, the hooks are constructed of a stainless steel or latex-free silicon so that they may be used in the sterile environment of the surgery. While such retractors as rakes or hooks are useful for certain types of injury, extreme care must be used to ensure that the retractor does not cause additional damage to the wound. In addition, use of the surgical retractor may require two, three, or more additional assistants to the physician, with appropriate training, in order to hold the retractor in the correct position so that the site of the surgery is more easily accessible to the physician. Other types of surgical retractors are inserted into the surgical site and then one or more arms are spread in order to open the insertion site for further access by the physician. These retractors are generally bulky, require substantial training and skill to operate, and user error may increase the difficulty and the time for the surgery. Traditional retraction using the above-mentioned retractors is recognized to cut-off circulation to the muscles and often results in post-operative pain and long-term degradation of muscle function.

Recently, minimally invasive techniques have been developed to reduce the intra-operative damage and reduce the post-operative recovery time. In minimally invasive surgery (MIS), a desired site is accessed through portals rather than through a significant incision. Various types of access portals have been developed for use in MIS. Many of the existing MIS access portals, such as those described in U.S. Pat. Nos. 4,573,488 and 5,395,317 issued to Kambin, can only be used for a specific procedure. Other prior art portals, such as that described in U.S. Pat. No. 5,439,464 issued to Shapiro, require multiple portals into the patient, adding complexity to the portal placement as well as obstructing the operating space.

### SUMMARY

According to one exemplary embodiment of the present system and method, a less invasive access port includes a retractor having a first member coupled to a second member. When the two retractor members are positioned for insertion into the tissue, the proximal ends are spaced apart from each other and the two distal portions are adjacent to each other. The retractor is then inserted into the tissue, adjacent the site for a desired medical procedure. The proximal ends of the two opposing retractors are then pushed together, which expands the distal portion to create a working space inside the tissue

In one exemplary embodiment, the less invasive access port is configured for use in minimally invasive surgery and allows for manipulation of the viewing angle into the working site in both an axial plane and a mediolateral plane. Further, the exemplary less invasive access port is configured to minimize muscle retraction. According to further aspects of the exemplary less invasive access port, sufficient light, irrigation, suction, and space for sundry medical instruments is provided through the access port.

According to principles of the present exemplary less invasive access port, the less invasive access port device includes a two-piece retractor wherein a proximal opening formed by the two-pieces decreases as a distal portion of the retractor expands. Further, a number of locking arms are formed on the proximal portion of the retractor. The locking arms may be secured outside of a wound by a latch when the device is in a retracted or open position. Further, a housing having a port there through is configured to engage the retractor, providing integrated light, irrigation, and suction mechanisms. Once engaged with the retractor, the housing is free to pivot within the two-piece retractor, thus providing access to the entire working site through the port. According to aspects of this embodiment, instruments and implants may be passed through the port and into the working space created by the two-piece retractor. According to aspects of one exemplary embodiment, visualization of the working site is preferably attained under direct vision.

Moreover, according to one exemplary embodiment, the present exemplary less invasive access port provides for a method of performing spinal surgery that includes percutaneously inserting one or more screws in a bony portion of a spine, placing a trocar onto the bony portion of the spine to provide access to the working site, inserting a retractor over the trocar down to the working site, inserting a cannula into the retractor, and opening the retractor to expose the working site. According to one exemplary embodiment, the insertion of the one or more screws, as well as insertion of the trocar, retractor, and the cannula are performed in the plane lateral to the multifidus in the fascial plane.

### BRIEF DESCRIPTION OF THE DRAWINGS

The accompanying drawings illustrate various exemplary embodiments of the present system and method and are a part of the specification. Together with the following description, the drawings demonstrate and explain the principles of the present system and method. The illustrated embodiments are examples of the present system and method and do not limit the scope thereof.

FIG. 1 is an isometric view of a less invasive access port, according to one exemplary embodiment.

FIG. 2 is a trocar used with the less invasive access port of FIG. 1, according to one exemplary embodiment.

FIG. 3 is a partial cut-away side view of a two-piece retractor inserted into a patient, according to one exemplary embodiment.

FIGS. 4A and 4B are a side view and a cross-sectional top view of a 2-piece retractor, respectively, according to one exemplary embodiment.

FIG. 5A is an isometric view showing a two-piece retractor having locking arms and a plurality of securing mechanisms, according to one exemplary embodiment.

FIGS. 5B and 5C are side views showing a two-piece retractor having ratcheting securing mechanisms, according to various exemplary embodiments.

FIG. 6 is a side view of a retractor having an optional soft tissue barrier, according to one exemplary embodiment.

FIGS. 7A and 7B is a side elevational view of a retractor portion of the less invasive access port assembly and a cross-sectional view of the assembly, respectively, according to one exemplary embodiment.

FIGS. 8A and 8B is a side elevational view of a retractor portion of the less invasive access port assembly and a cross-sectional view of the assembly, respectively, according to one exemplary embodiment.

FIG. 9A is a plan isometric view of a cannula assembly, according to one exemplary embodiment.

FIG. 9B is an isometric view of a cannula assembly having a leyla arm attachment thereon, according to one exemplary embodiment.

FIG. 9C is a bottom isometric view of the cannula assembly of FIG. 9A, according to one exemplary embodiment.

FIG. 9D is an isometric view of the cannula sleeve, according to one exemplary embodiment.

FIG. 10A is an isometric view of a two piece retractor slid over a trocar, according to one exemplary embodiment.

FIG. 10B is an isometric view of a cannula assembly introduced over a trocar to engage a two-piece retractor shown in FIG. 3, according to one exemplary embodiment.

FIG. 10C is an isometric view of the less invasive access port in a deployed position prior to removal of the trocar, according to one exemplary embodiment.

FIG. 11 is a flow chart illustrating a method for performing spinal surgery using the present less invasive access port, according to one exemplary embodiment.

FIG. 12 is a top view illustrating the insertion of a pedicle screw in the fascial plane lateral to the multifidus, according to one exemplary embodiment.

FIG. 13 is a side elevational view of yet another embodiment of the less invasive access port illustrating a mediolateral pivot ability, according to one exemplary embodiment.

FIGS. 14A-14E are side elevational views of a two-piece retractor in various deployed, undeployed and positions there between during a spinal surgery procedure, according to one exemplary embodiment.

FIGS. 15A-15C are side elevational views of a further embodiment of a two-piece retractor in a deployed and an undeployed position having a cut-away viewing tube thereon, according to one exemplary embodiment.

Throughout the drawings, identical reference numbers designate similar but not necessarily identical elements.

### DETAILED DESCRIPTION

The present specification describes a system and a method for performing spinal surgery using minimal invasive surgery (MIS) techniques. Further, according to one exemplary embodiment, the present specification describes a less invasive access port that allows for mediolateral pivot of a cannula member while maintaining a retractor locking mechanism outside the wound. Additionally, the exemplary less invasive access port device described herein provides integrated light, suction, and irrigation capabilities, without interfering with the operational access port. The functionality of the less invasive access port described herein allows for a surgical method wherein any number of pedicle screws are inserted prior to the insertion of the less invasive access port. Moreover, the present exemplary MIS technique includes insertion of the pedicle screw(s) and the less invasive access port in the fascial plane lateral to the multifidus, thereby greatly reducing damage to soft tissue during surgery. Further details of the present exemplary system and method will be provided below.

By way of example, pedicle screw systems may be fixed in the spine in a posterior lumbar fusion process via minimally invasive surgery (MIS) techniques. The systems are inserted into the pedicles of the spine and then interconnected with rods to manipulate (e.g., correct the curvature, compress or expand, and/or structurally reinforce) at least portions of the spine. Using the MIS approach to spinal fixation and/or correction surgery has been shown to decrease a patient's recovery time and reduce the risks of follow-up surgeries.

The ability to efficiently perform spinal fixation and/or correction surgeries using MIS techniques is enhanced by the use of the less invasive access port and its associated surgery method provided in accordance with the present exemplary systems and methods, which systems and methods provide a number of advantages over conventional systems, as will be detailed below.

In the following description, for purposes of explanation, numerous specific details are set forth in order to provide a thorough understanding of the present system and method for a less invasive access port system. It will be apparent, however, to one skilled in the art that the present method may be practiced without these specific details. In other instances, well-known structures associated with the less invasive access port have not been shown or described in detail to avoid unnecessarily obscuring descriptions of the embodiments. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

Exemplary Overall Structure

While the present system and method may be practiced by or incorporated into any number of systems, the present system and method will be described herein, for ease of explanation only, in the context of a less invasive access portal for use in orthopedic spinal surgery; providing a channel to the underlying bony structures of the spine while minimizing trauma to the overlying tissues. According to aspects of the present exemplary system and method, the less invasive access portal is able to minimize the need for muscle retraction. Additionally, according to one exemplary embodiment, the less invasive access portal provides sufficient light, irrigation, suction and space for sundry medical instruments. The features and advantages of the exemplary systems and methods will be set forth in the description which follows, and in part will be apparent from the description.

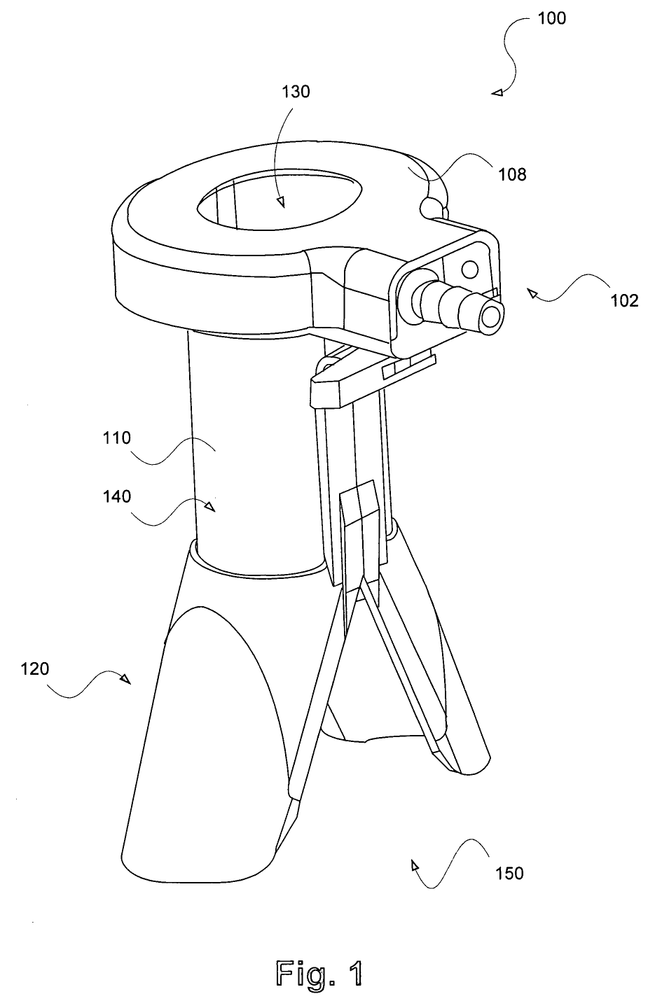

FIG. 1 shows an assembled less invasive access port device (100) in a deployed position, according to one exemplary embodiment. As shown, the exemplary less invasive access port device (100) includes a two-piece retractor (120) having a proximal (140) and a distal end (150). Additionally, a cannula (110) is coupled to the proximal end (140) of the two-piece retractor (120). An inner wall of the cannula (110) defines an access port (130). According to one exemplary embodiment, instruments and implants may be passed through the access port (130) defined by the cannula (110) and into a working space created by the two-piece retractor (120). Further, as illustrated in FIG. 1, the cannula portion (110) of the less invasive access port device (100) includes integrated interfaces (102) for light, irrigation and suction. According to the exemplary embodiment shown in FIG. 1, a housing (108) forms a collar around a top of the cannula (110) and houses the light, irrigation and suction interface (102), as well as the light, irrigation, and suction channels. According to one exemplary embodiment described in further detail below, the cannula (110) is rotatably coupled to the two-piece retractor (120) such that the cannula may be rotated in both axial and mediolateral directions once a trocar and k-wire are removed. The ability for the cannula (110) to achieve both axial and mediolateral rotation within the two-piece retractor (120) provides access to the entire working site defined by the two-piece retractor (120). According to one exemplary embodiment, visualization of the working site is attained under direct vision. Further details of each component of the less invasive access port device (100), their assembly, and the tools used in conjunction therewith will be provided below with reference to FIGS. 1-10C.

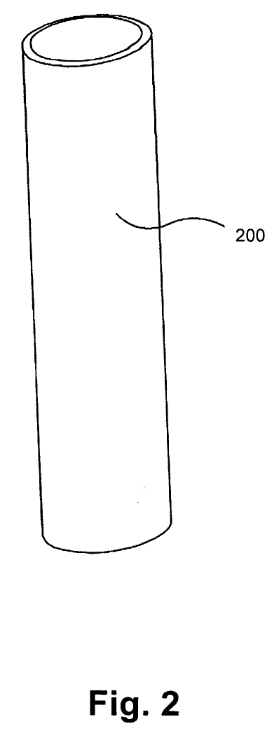

As mentioned above, the exemplary less invasive access port device (100) may be slideably positioned into a work area by the use of a trocar. FIG. 2 illustrates an exemplary trocar (200) for use with the less invasive access port device (100) of FIG. 1. In operation, a k-wire may be initially inserted into the soft tissues. Any number of pedicle screws may then be percutaneously inserted into a desired bone mass. The trocar (200) may then be placed over the k-wire to dilate the soft tissues and provide access to a desired working site. As used herein, the trocar (200) may be any number of stylets used for exploring or dilating tissue. According to one exemplary embodiment, the trocar (200) includes a triangular point on one end. However, the point of the trocar (200) used in connection with the present exemplary less invasive access port device (100) may assume any number of geometric profiles.

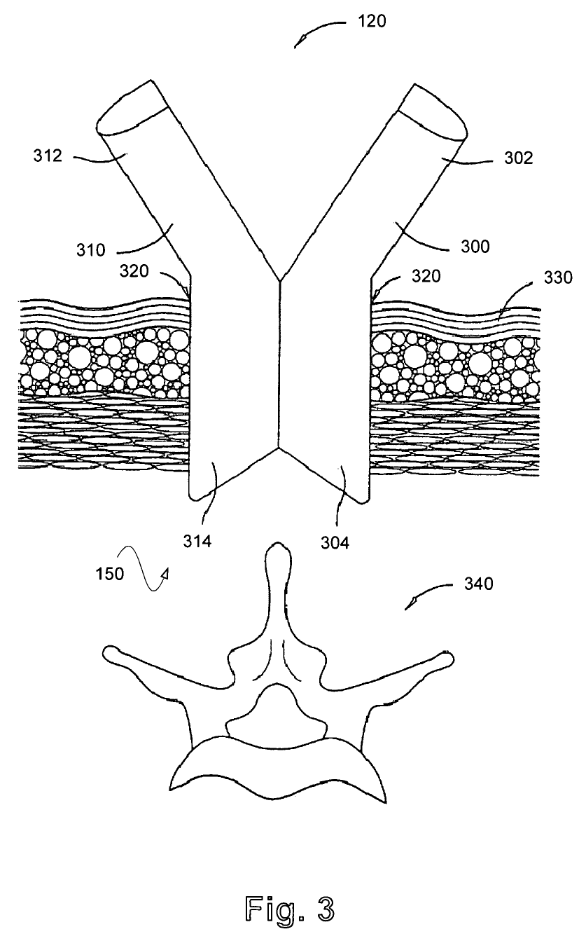

Continuing with the exemplary components of the less invasive access port device (100) of FIG. 1, the bottom portion of the device includes a two-piece retractor (120). According to one exemplary embodiment, the two-piece retractor (120) of the present exemplary less invasive access port (100) includes a proximal end (140) and a distal end (150), wherein expansion of the distal end (150) of the two-piece retractor (120) causes a compression of the proximal end (140) of the retractor, and vice versa. Particularly, FIG. 3 illustrates an exemplary two-piece retractor (120) that operates as described above. Specifically, as illustrated in the exemplary embodiment of FIG. 3, the two-piece retractor (120) includes a first member (300) and a second member (310) rotatably coupled at a central location. The first member (300) includes a top portion (302) and a bottom portion (304). Similarly, the second member (310) includes a bottom portion (314) and a top portion (312). Each of the retractor members (300, 312) are individual members that may or may not be coupled to each other.

When inserted into an opening (320) in the skin (330), the top portions (302, 312) may be separated as illustrated in FIG. 3, causing the bottom portions (304, 314) to be closely positioned, to be minimally invasive. Once inserted, the two-piece retractor (120) may be actuated to provide workable access to a vertebra (340) or other desired structure.

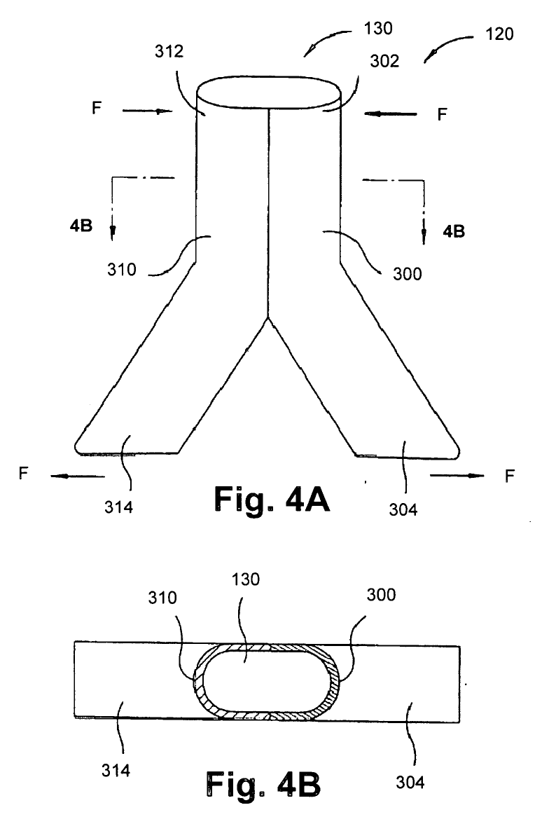

FIG. 4A illustrates the retractor (120) performing a retraction function, which causes the bottom portions (304, 314) to spread apart from each other. As the bottom portions (304, 314) spread apart from each other, muscle and tissue is lifted from the desired medical site, allowing vision and access to the desired medical site. As illustrated by the arrows in FIG. 4A, the expansion of the bottom portions (304; 314) of the two-piece retractor (120) causes a contraction of the top portions (302, 312). Consequently, the top portions (302, 312) of the two-piece retractor (120) join to form an access port (130). The resulting access port (130) provides access to the desired medical site. The desired medical site may be any acceptable medical site, such as a vertebra (340) or other location to which a surgeon desires to have clear and clean access.

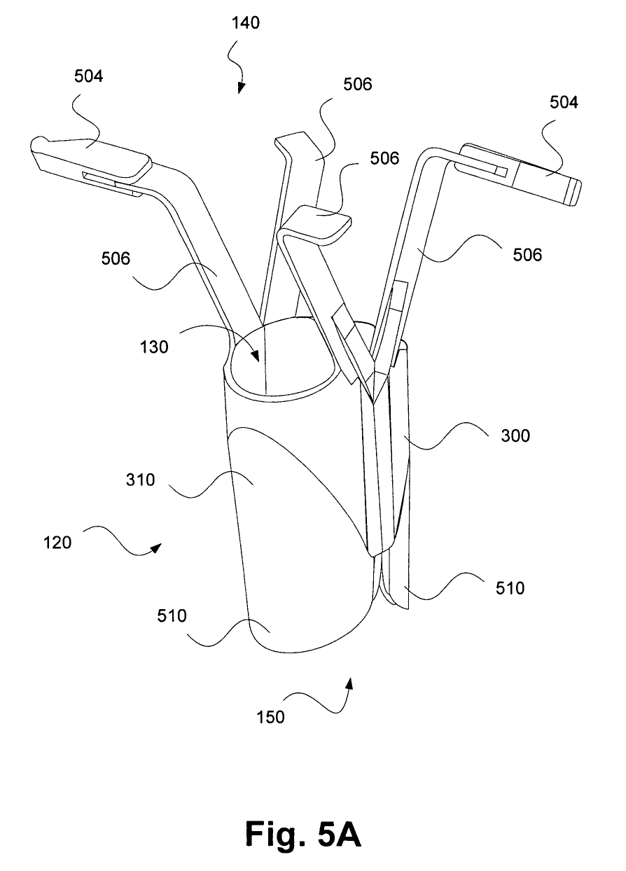

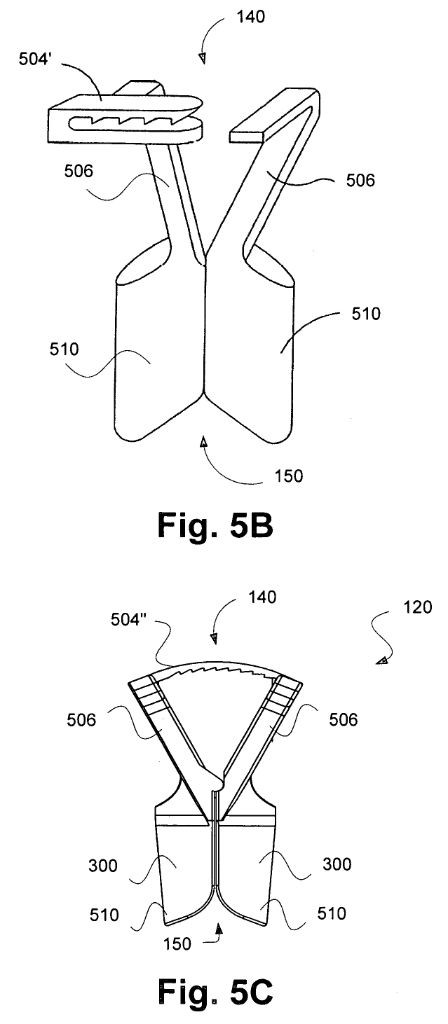

FIG. 5A illustrates an exemplary two-piece retractor (120) that may be used with the present less invasive access port device (100; FIG. 1), according to one exemplary embodiment. As illustrated in FIG. 5A, the two-piece retractor (120) includes top portions made up of moveable locking arms (506) and a number of securing devices (504) for retaining the locking arms (506). Further, the bottom portions of the exemplary two-piece retractor (120) include retractor blades (510). Similar to the two-piece retractor (120) of FIG. 3, the proximal portion (140) and the distal portion (150) operate in opposing directions. Specifically, when the moveable locking arms (506) are in a separated state, the retractor blades (510) are joined. Conversely, when the moveable locking arms (506) are brought together, the retractor blades (510) are separated to provide access to a desired medical site. In order to maintain access to the desired medical site, the moveable locking arms (506) may be secured in their joined position by the securing devices (504), assuring that the access to the desired medical site will be maintained. According to the exemplary embodiment illustrated in FIG. 5A, the securing device (504) may be a compliant male arm that snaps into a rigid female arm. Alternatively, according to various exemplary alternative embodiments, the securing device (504) may include, but is in no way limited to a latch, a ratcheting latch, a clasp, a hook, a catch, a cam lever, or the like.

FIG. 5B illustrates a retractor (120) including an alternative securing mechanism (504′), according to one exemplary embodiment. Specifically, FIG. 5B shows a two-piece retractor (120) having a ratcheted retaining mechanism (504′) affixed to at least one arm (506) of the retractor. According to the exemplary embodiment illustrated in FIG. 5B, a second locking arm (506) of the retractor (120) may include a protrusion configured to be securely received by the retaining mechanism (504′) when the retractor is in a deployed position with the retractor blades (510) separated. One advantage of the exemplary two-piece retractor (120) over traditional retractors is that by designing the retractor so that the proximal portion (140) and the distal portion (150) operate in opposing directions, the locking mechanism or securing device may be positioned on the proximal portion of the retractor (120). Consequently, in contrast to traditional retractors, the locking mechanism will be located outside of the wound during a medical procedure, providing convenient access to a surgeon for deployment and/or retraction. Additionally, the ability to draw in the retractor blades (510) after deployment allows the present two-piece retractor (120) to be re-usable.

Similarly, FIG. 5C illustrates yet another alternative securing mechanism (504″), according to an alternative embodiment. As shown, the ratcheting securing mechanism (504″) may be used to secure the position of the locking arms (506), and consequently the retractor blades (510) in any number of deployed stages.

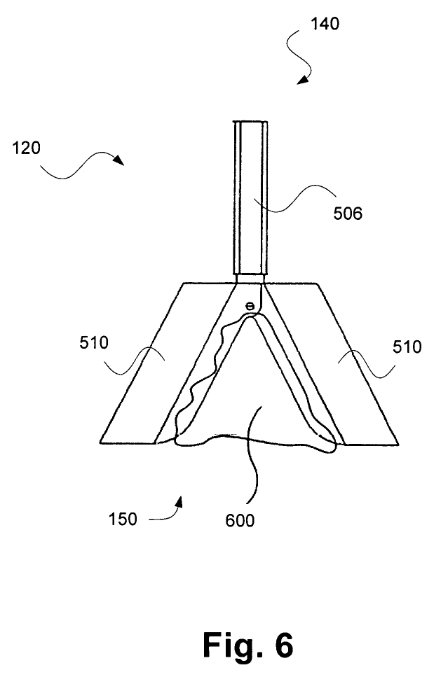

FIG. 6 shows yet another embodiment of the two-part retractor (120) having an optional soft tissue barrier (600). According to one exemplary embodiment, a flexible material may be added to the retractor blades (510) such that when the retractor blades are deployed, the open space between the blades is occupied by the soft tissue barrier (600). The soft tissue barrier (600) may be added between the retractor blades (510), according to one exemplary embodiment, to ensure that soft tissue does not herniate into the working channel when the retractor blades (510) are deployed.

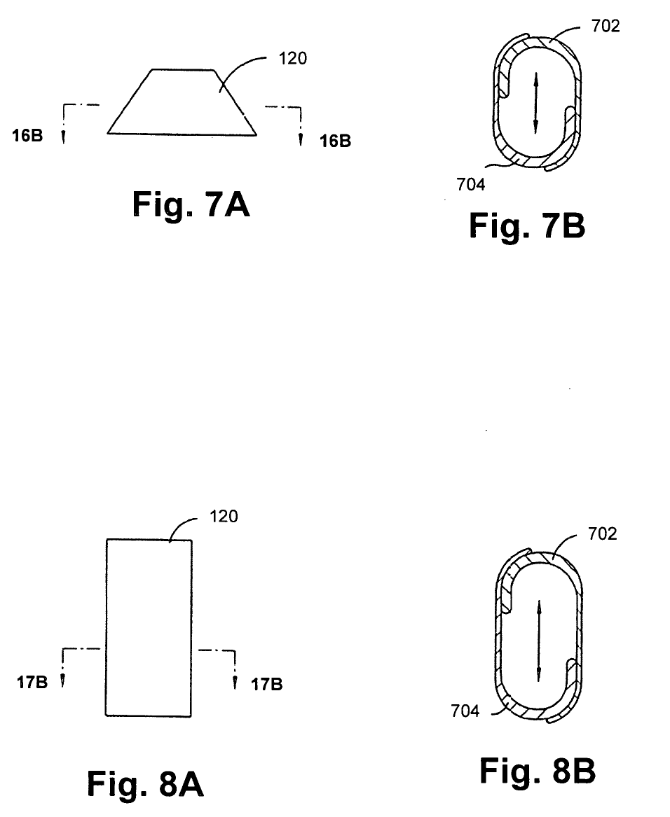

Alternatively, rather than incorporating a soft tissue barrier (600), the retractor blades (510) may be designed to provide an enclosed space when deployed. Particularly, FIG. 7A is a side elevational view of an exemplary two-piece retractor (120) portion of the less invasive access port assembly (100; FIG. 1) in an open position. Similarly, FIG. 7B is a cross-sectional view of the assembly. According to this exemplary embodiment, the retractor blades (510) may include a first stiff retractor portion (702) and a first flexible retractor portion (704) coupled in an overlapping configuration shown in FIG. 7B. Similarly, FIG. 8A is a side elevational view of the two-piece retractor (120) of the less invasive access port assembly (100; FIG. 1) in a closed position and FIG. 8B is a cross-sectional view of the assembly in the closed position, according to one exemplary embodiment. According to this exemplary embodiment, the two-piece retractor (120) has a stiff retractor portion (702) and a flexible retractor portion (704). The flexible retractor portion (704) extends around the opposing retractor to enclose the cavity. When opened, the flexible retractor portion (704) slides over the opposing stiff retractor portion, allowing the two retractor portions (702, 704) to spread apart while maintaining an enclosed space.

While the retractor blades (510) of the exemplary two-piece retractor (120) have been described above and illustrated in the Figures as having a particular shape, the retractor blades (510) of the two-piece retractor (120) may assume any number of shapes, and may be made of any number of materials to satisfy a desired surgical purpose.

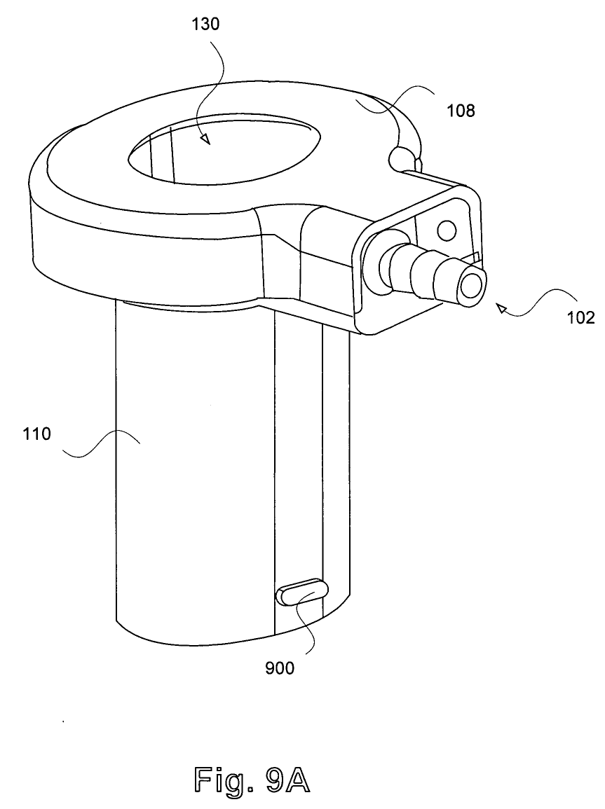

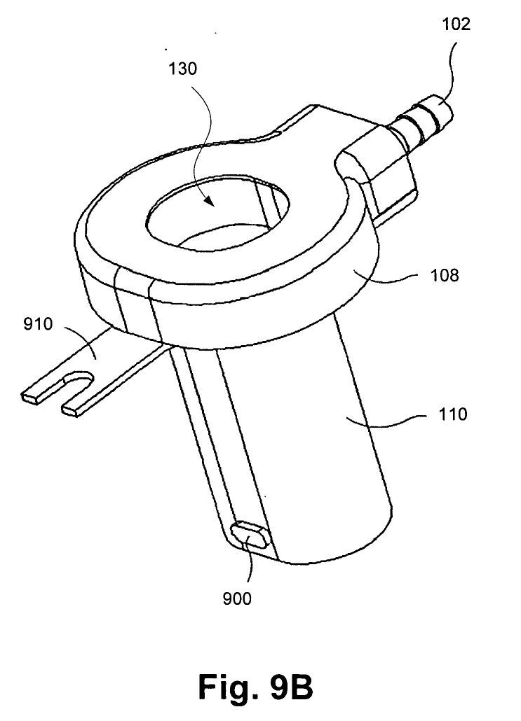

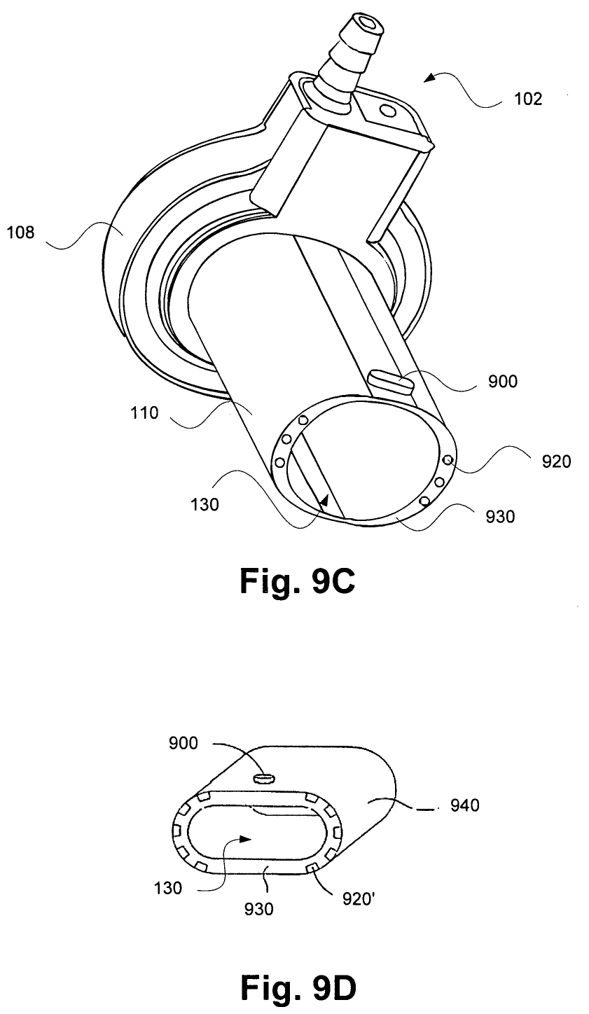

Continuing with the components of the exemplary less invasive access port device (100; FIG. 1), FIG. 9A is a perspective view illustrating an exemplary cannula assembly (110) prior to engagement with the two-piece retractor (120; FIG. 1). As shown, the exemplary cannula defines an access port (130), includes a housing (108) on a proximal end of the cannula assembly (110), includes a boss (900) formed on a distal exterior surface of the cannula assembly. According to the exemplary embodiment illustrated in FIG. 9A, the housing (108) includes integrated interfaces (102) for fiber optic lights, irrigation, and suction. According to one exemplary embodiment illustrated in FIG. 9A, the access port (130) defined by the body of the cannula assembly (110) is sufficiently large and of an appropriate geometry to allow for the passage of a number of operating tools to access an identified surgical location. Additionally, the access port (130) may also provide an optical inspection portal, allowing a surgeon to visually inspect the identified surgical location without the use of optical cameras and the like.

The pivot boss (900) formed on the distal portion of the cannula assembly (110) is configured to allow the cannula assembly (110) to be received and captured by a corresponding female orifice (not shown) formed on the inner wall of the two-piece retractor (120). According to this exemplary embodiment, the pivot boss (900) is sufficiently protruding beyond the cannula assembly (110) to be captured by the corresponding female orifice while allowing the cannula assembly to pivot in any direction relative to the two-piece retractor (120) without causing the release of the cannula assembly. According to various exemplary embodiments, the boss (900) can be a relatively oval tab as shown in FIG. 9A, or the pivot boss (900) may assume the shape of any other suitable retaining mechanism for securing the cannula assembly (110) to the two-piece retractor (120) while allowing pivotal movement there between.

FIG. 9B shows an alternative embodiment of a cannula assembly having a leyla arm attachment (910) coupled thereto. The attachment (910) serves as a mount for attachment of the cannula assembly (110) to a positioning arm during an operation. In alternative embodiments, mounts of various size and configuration as are known in the art and could be added to the cannula assembly. As illustrated in FIG. 9B, the boss (900) may be formed on opposing sides of the cannula assembly (110) to facilitate multi-axial rotation of the cannula assembly (110) within the two-piece retractor (120).

FIG. 9C is a bottom isometric view of the cannula assembly (110), according to one exemplary embodiment. As illustrated in FIG. 9C, a number of channels (920) are contained in the cannula wall (930) connecting the work site with the collar (108) at a proximal end of the cannula assembly (110). According to one exemplary embodiment, aspiration and irrigation of the work site is accomplished through the channels (920) or passages in the distal face of the cannula assembly (110). The integrated interfaces (102) are contained on the collar (108) and connect to the channels (920) to support the aspiration and irrigation at the work site. Additionally, according to one exemplary embodiment, light can be supplied to the cannula assembly (110), and consequently the work site, through a fiber-optic cable, similar to that used with surgical headlamps. According to one exemplary embodiment, the fiber optic cables are truncated at the distal face of the cannula assembly (110). According to this exemplary embodiment, light from a fiber optic cable will pass down the wall of the cannula assembly (110), as it would a fiber-optic cable, to illuminate the work site.

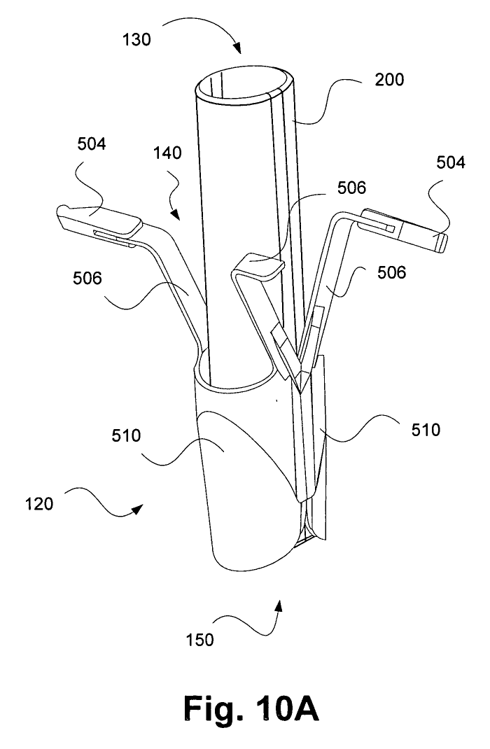

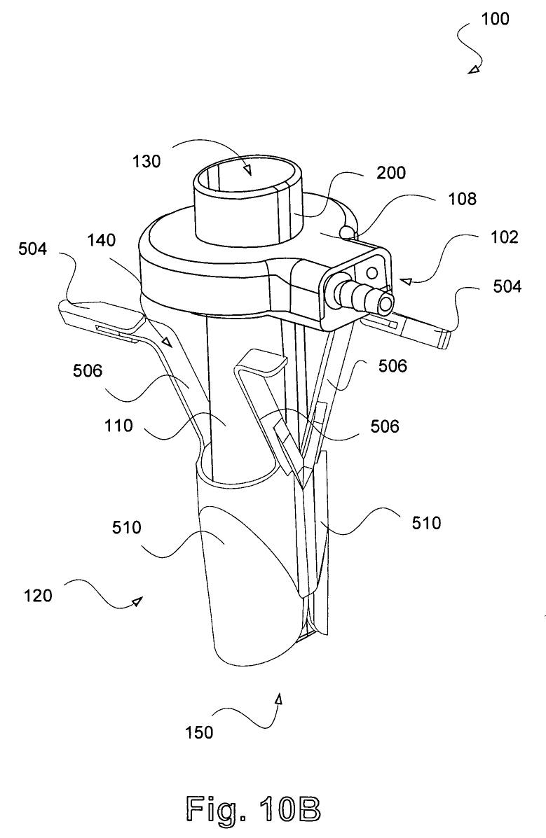

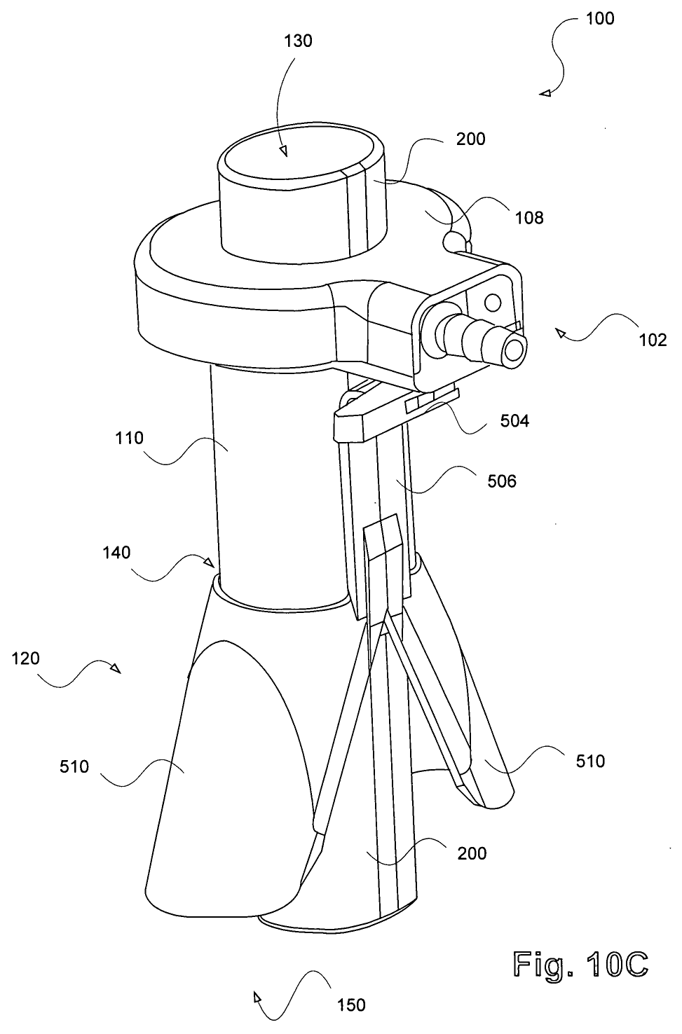

While the channels (920) may be drilled or otherwise formed in the cannula wall (930), FIG. 9D illustrates an alternative embodiment of the cannula wall (930). According to the exemplary embodiment illustrated in FIG. 9D, the cannula sleeve (940) includes a cannula wall (930) defining an access port (130). The outer surface of the cannula wall (930) includes a plurality of ridges or fins defining slots (920′) in the exterior cannula wall (930). Further, a cannula sleeve (940) or sheath is formed over the outside of the cannula wall (930) to seal the fins or slots (920′) contained on an outside surface of the cannula wall (930). The slots (920′) contained on the outside surface of the cannula wall (930) may be ridges, grooves, channels, fins or the like. The slots (920′) provide a passage for aspiration, the placement of fiber optic filaments as a light source, video feed, or the like. In accordance with aspects of the present exemplary embodiment, the cannula assembly (110) may be made out of a light transmitting material to channel light into the working space through the walls of the cannula. Assembly and deployment of the exemplary less invasive access port device (100; FIG. 1) will now be described with reference to FIGS. 10A through 10C.

As mentioned previously, a k-wire may be inserted, with the aid of a fluoroscope, into a desired working space. Any number of pedicle screws may then be percutaneously inserted into a desired bone mass. A trocar (200) may then be placed over the k-wire to dilate the soft tissues and provide access to a desired working site. With the trocar appropriately placed, a two-piece retractor (120) can be introduced over the trocar (210) and down to the working site (not shown). As illustrated in FIG. 10A, the two-piece retractor (120) in its un-deployed configuration retains the retractor blades (510) adjacent to one another, forming a channel. The trocar (200) can be received within the distal opening of the channel and the two-piece retractor (120) may then be slid down the trocar (200) in its undeployed state until the distal portion (150) of the retractor is in a desired working space.

With the two-piece retractor (120) correctly positioned in the desired working space, the cannula assembly (110) may also be introduced over the trocar (200) until it engages the two-piece retractor. FIG. 10B illustrates an exemplary cannula assembly introduced over the trocar (200). As illustrated, the two-piece retractor (120) has not been deployed, and thus the locking arms (506), remain in an open, unlocked position. As mentioned previously, the cannula assembly includes a number of bosses (900; FIG. 9C) which may engage mating reception recesses within the two-piece retractor (120) to couple the cannula assembly (110) to the retractor (120), while maintaining the ability to have axial and mediolateral rotation. As shown in FIG. 10B, the trocar (200) is received through the access port (130) of the cannula assembly (110).

FIG. 10C shows the less invasive access port device (100) in a deployed position prior to removal of the trocar (200) from the assembly. As shown, the locking arms (506) of the two-piece retractor (120) are drawn together, thus closing the top or proximal end (140) of the two-piece retractor about the cannula assembly (110). As the locking arms (506) of the two-piece retractor (120) are drawn together, the distal end (150) of the retractor opens to further dilate the soft tissues at the working site. The retractor (120) is secured in a deployed position with retaining mechanism (504) affixed to each set of locking arms (506). With the two-piece retractor (120) in a deployed position, the trocar (200) may be removed and the working site may be manipulated.

According to one exemplary embodiment, the two-piece retractor (120) can be diametrically expanded after it is deployed. This will increase the working area/channel within the retractor. Any appropriate expanding instrument could be used.

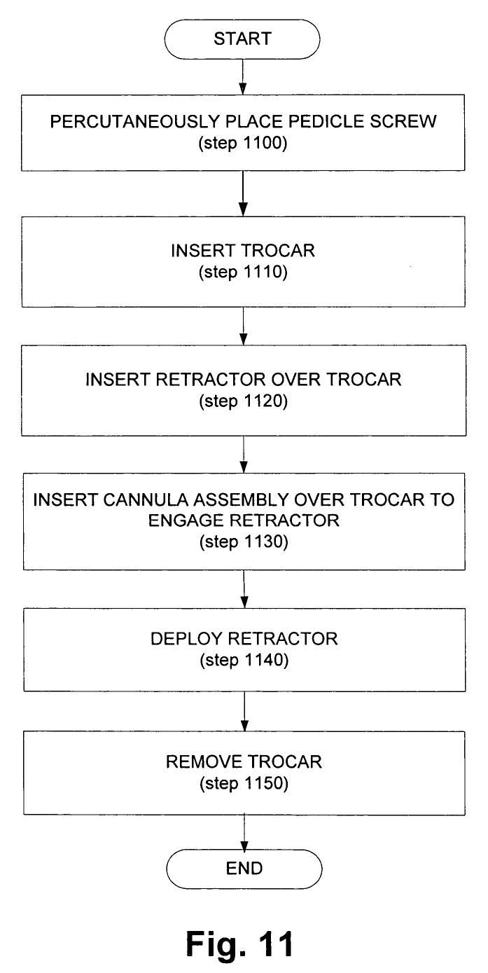

Further details of the implementation and operation of the less invasive access port device (100) will be provided below with reference to FIGS. 11 through 13.

Exemplary Implementation and Operation

FIG. 11 illustrates an exemplary method for using the present exemplary less invasive access port device (100) to access a desired work site on a patient's spine. As illustrated in FIG. 11, the exemplary method begins by first percutaneously placing one or more pedicle screws in vertebra (step 1100). With the pedicle screws in place, a trocar or other dilating device may be inserted at the location of the pedicle screw (step 1110). With the trocar in place, a two-piece retractor is slideably inserted over the trocar (step 1120), followed by the insertion of a cannula assembly over the trocar to engage the two-piece retractor (step 1130). With the less invasive access port device (100; FIG. 1) assembled, the retractor may then be deployed (step 1140) followed by the removal of the trocar (step 1150). Further details of each step of the above-mentioned method will be provided below with reference to FIGS. 11 through 13.

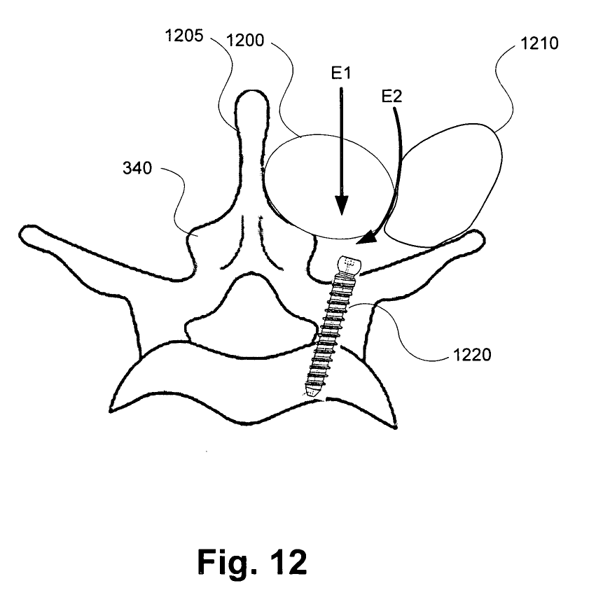

As mentioned above, the present exemplary method includes inserting one or more pedicle screws in a patient's vertebra (step 1100) prior to the insertion of a trocar or cannula. According to one exemplary embodiment, the percutaneous insertion of one or more pedicle screws (step 1100), the insertion of the trocar (step 1120), and the insertion of the retractor over the trocar (step 1130) is performed in the plane lateral to the multifidus. As illustrated in FIG. 12, the lumbar vertebra (340) have a number of muscle groups that run on top of the vertebra. As shown in FIG. 12, the multifidus (1200) is located adjacent to the spinous process (1205). The longissimus muscle group (1210) is positioned lateral to the multifidus (1200). Current MIS approaches insert pedicle screws and their associated hardware through an entry path that traversed the multifidus muscle group (1200), as illustrated by E1. This technique unnecessarily damages soft tissue, resulting in pain and increased rehabilitation for the patient. According to the present exemplary embodiment, the entry path illustrated by E2 is used for the insertion of the pedicle screw, a trocar, or a cannula.

Specifically, insertion of one or more pedicle screws in a patient's vertebra (step 1100) includes performing a blunt dissection in the plane lateral to the multifidus (1200) approaching the area of the transverse process where it reaches the lateral aspect of the facet joint. Then, under fluoroscopic guidance, a screwdriver, screw/sleeve assembly with or without a sleeve (not shown) can be used to place the pedicle screw (1220) in the vertebra (340).

With the pedicle screw(s) (1220) in place, a trocar or other sleeve may be inserted, in the plane lateral to the multifidus, to the location of the pedicle screw(s) (step 1110). Insertion of the trocar dilates the soft tissue, allowing the formation of a working space. With the trocar appropriately placed, the two-part retractor (120; FIG. 1) is placed over the trocar and slideably inserted into the working space (step 1120). As mentioned previously, when the two-part retractor (120; FIG. 1) is positioned within the working space, the locking arms (506; FIG. 5A) are at least partially located outside the wound. This allows the two-part retractor to be easily locked in a deployed position.

With the retractor properly placed, the cannula assembly may be placed over the trocar and engaged with the retractor (step 1130) followed by deployment of the retractor (step 1140). According to one exemplary embodiment, the deployment of the retractor and engagement of the cannula assembly with the retractor may be performed in any order. According to one exemplary embodiment, when the two-part retractor is deployed (step 1140), the muscles surrounding the working space are retracted. Prior to deploying the retractor, a series of Cobb elevators and other instruments could be used to subperiosteally dissect the muscle off the facet joints and lamina and spinous processes creating a working space for the retractor to be deployed in.

When the retractor is deployed in the working space, the trocar and any other sleeves may be removed from the access port of the less invasive access port device (step 1150). Once removed, the working space may be accessed for performing decompression, discectomy, interbody fusion, partial facetectomy, neural foraminotomy, facet fusion, posterolateral fusion, spinous process removal, placement of interspinous process distractors, or facet replacement, pedicle replacement, posterior lumbar disc replacement, or any one of a number of other procedures.

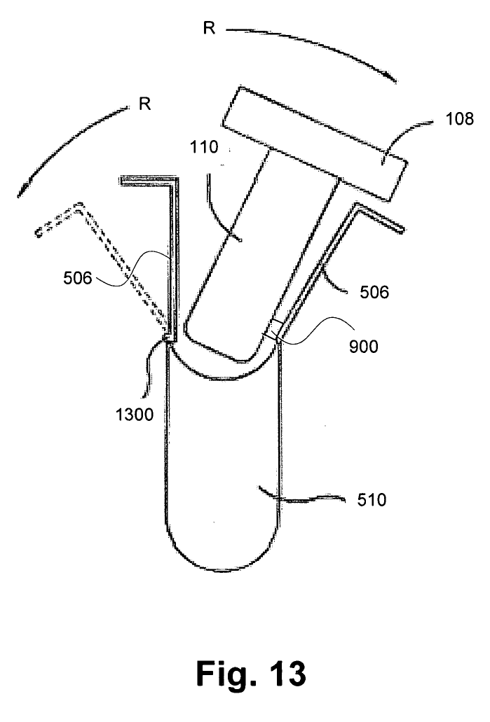

Performance of the various procedures via the access port (130; FIG. 1) is facilitated by the rotational freedom provided by the present less invasive access port device (100; FIG. 1). FIG. 13 illustrates a mediolateral motion of the cannula assembly (110) within the two-piece retractor (120). Specifically, the mediolateral motion of the cannula assembly (110) within the two-piece retractor (120) may be facilitated by a number of elements. First, the boss (900) formed on the distal exterior surface of the cannula assembly (110) can mate with a recess formed on the inner surface of the retractor (120) to retain the cannula assembly (110) within the retractor during mediolateral pivoting. Additionally, the cannula assembly (110) may pivot about a hinge point (1300) of the retractor (120) to provide a multidirectional viewing window. A hinge (1300) between the arm (506) and the retractor blades (510) allows side-to-side movement of the cannula cannula assembly. According to an alternative embodiment, the arms (506) can be attached to the retractor (120) by a flexible material or a compliant section to allow the arms to flex when the cannula is moved side to side, thus allowing the cannula a full range of motion. Alternatively, the arms may be detachable or may fold down in a retracted position.

### Alternative Embodiments

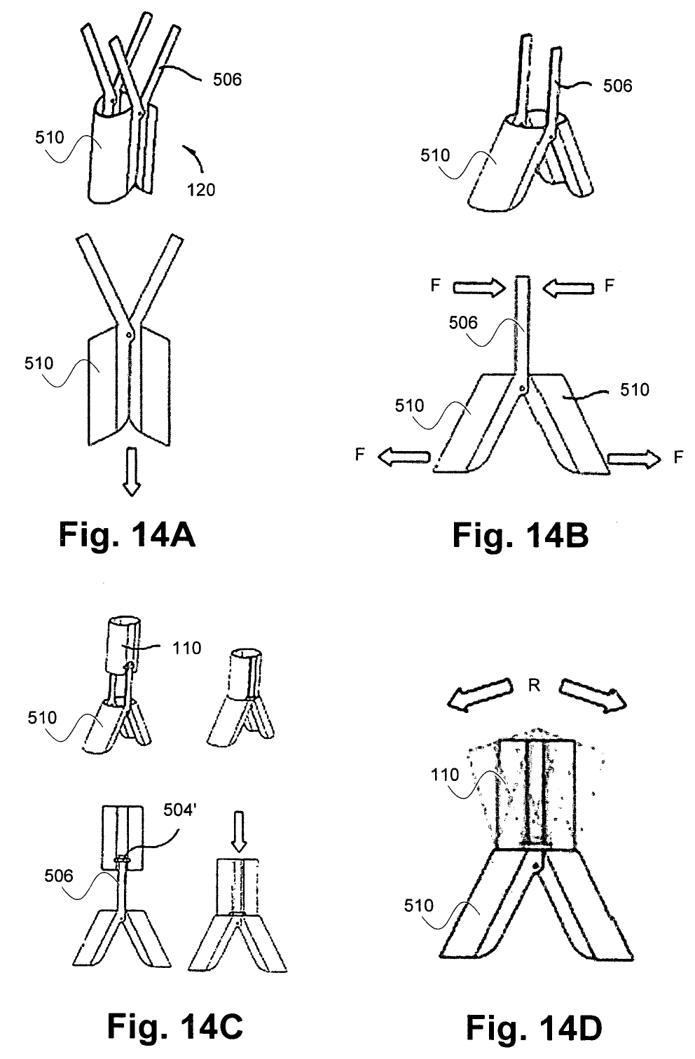

FIGS. 14A-14D show an alternative embodiment of the less invasive access port assembly device (100) in various degrees of deployment. The device (100) includes an alternative retaining mechanism (504′) for retaining the retractor (120) in a deployed position. Specifically, the retaining mechanism (504′) is contained on the cannula (110), which is not aligned with the retractor until the retractor (120) has been deployed. FIG. 14D illustrates how the interface between the retractor (120) and the cannula (110) is a hinged interface that allows the cannula to be moved about a vertical axis to allow full viewing of the work site.

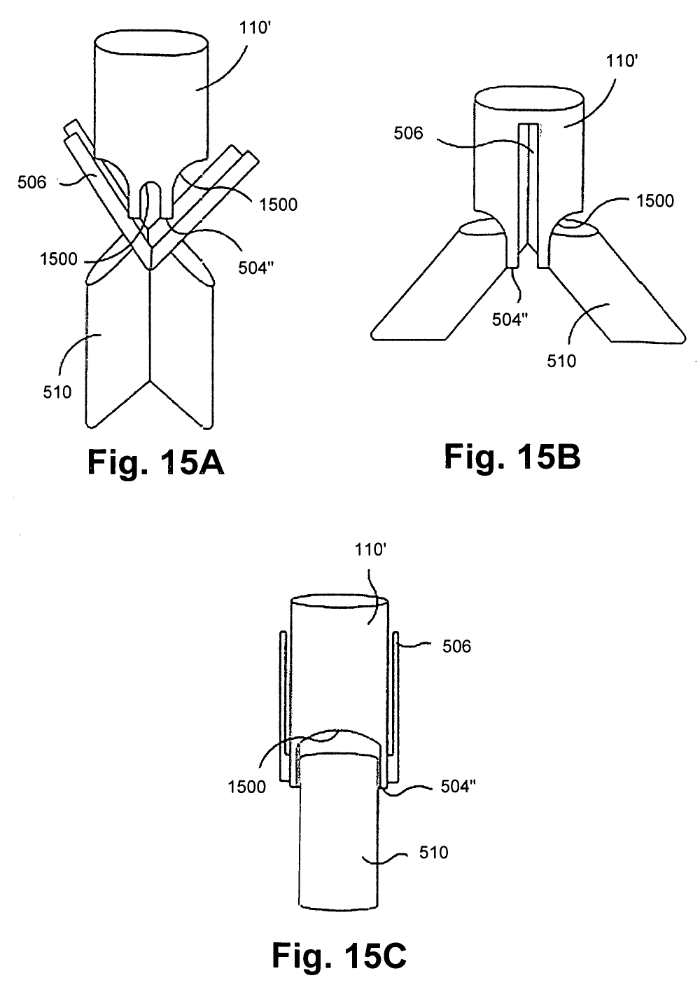

FIGS. 15A-1 5C illustrate a further embodiment of a two-piece retractor (120) in a deployed and an un-deployed position having a cut-away viewing tube (110′) thereon. The cut-outs (1500), allow the tube (110′) to slide over a hinging point between the arms (506) and the retractor blades (510). The cut-out (1500) with tabs (504″) on either side of the arms (506) thus serve as a securing mechanism for keeping the retractor in a full deployed position (and the arms in a closed position).

Further advantages of the present exemplary system include the variety of materials, including composites, plastics and radio-opaque materials, that the cannula and retractor can be made from. Existing MIS access ports are made of metal, which has several shortcomings: metal conducts electricity which can cause arcing from an electrocautery device and thus unwanted stimulation of the nerves; metals are reflective and produce an environment that is difficult to clearly view the surgical site; metals are radio-opaque and make intra-operative x-ray difficult. Alternative materials that are partially radio-opaque would provide for optimal intra- operative x-ray. The geometry and structural integrity of the prior art does not allow for the use of alternative materials.

In conclusion, the present exemplary systems and methods allow for a surgeon to manipulate the viewing angle of the less invasive access port into the working site in a transverse plane. Manipulation of a port medially and laterally facilitates: decompression of the neural elements; simple access to the contralateral side of the spine, eliminating the need to place a tube through the skin on that side; access to the transverse process on the ipsalateral side for a posterolateral fusion, and generally simplifies a surgical procedure by increasing the surgeon's viewing of the surgical site. Further, the present exemplary systems and methods allow for the retraction of muscles rather than the distal lifting of muscles during procedures. Additionally, the present exemplary system positions the arm securing mechanism outside of the wound where it may be readily accessed by the surgeon.

Moreover, the present system and method do not require the additional use of a light source, a suction device, and an irrigation device because these items are integral to the construction of the less invasive access port device. Existing MIS access ports require the additional use of a light source, a suction device, and an irrigation device, all of which decrease the space left for surgical instruments and for viewing of the surgical site.

The preceding description has been presented only to illustrate and describe the present method and system. It is not intended to be exhaustive or to limit the present system and method to any precise form disclosed. Many modifications and variations are possible in light of the above teaching.

The foregoing embodiments were chosen and described in order to illustrate principles of the system and method as well as some practical applications. The preceding description enables others skilled in the art to utilize the method and system in various embodiments and with various modifications as are suited to the particular use contemplated. It is intended that the scope of the present exemplary system and method be defined by the following claims.

What is claimed is:

1. A less invasive access port, comprising:
a retractor including a first retractor blade, a second retractor blade, at least one arm extending from said first retractor blade, and at least one arm extending from said second retractor blade;
a cannula having walls, said cannula sized to slideably couple to said retractor.

2. The less invasive access port of claim 1, further comprising a retaining member coupled to one of said arms, said retaining member being configured to releasably retain said retractor in a deployed position.

3. The less invasive access port of claim 2 wherein said retaining member comprises a ratcheting latch affixed to one of said arms.

4. The less invasive access port of claim 1, further comprising a channel in the wall of the cannula fluidly connecting a top portion of said cannula to a bottom portion of said cannula, wherein fiber optic strands are disposed in said cannula channels.

5. The less invasive access port of claim 4 wherein said cannula walls are light transmitting.

6. The less invasive access port of claim 4, further comprising a housing fluidly connected to said cannula channels, said housing including at least one integrated interface port.

7. The less invasive access port of claim 1 further comprising:
a boss disposed on said bottom portion of said cannula;
wherein said boss is configured to mate with an opening in said retractor to pivotably connect said cannula to said retractor.

8. The less invasive access port of claim 7, wherein said cannula is configured to pivot along two axes.

9. The less invasive access port of claim 8, wherein said arms are flexibly connected to said first and second retractor blade to allow said cannula to pivot in both an axial and a mediolateral direction.

10. The less invasive access port of claim 2 wherein said retaining member is configured to be outside a wound when said retractor is deployed in said wound.

11. A retractor comprising:
a first retractor blade;
a second retractor blade:
at least one arm extending from a top of said first retractor blade;
at least one arm extending from a top of said second retractor blade;
and a retaining member coupled to one of said arms, said retaining member being configured to releasably retain said retractor in a deployed position;
wherein said retaining member is configured to be outside a wound when said retractor is deployed inside said wound.

12. The retractor of claim 11, wherein said retaining member comprises a ratcheting latch affixed one of said arms.

13. The retractor of claim 11, wherein said first and second retractor blades expand when said arms are contracted.

14. A less invasive access port, comprising:
a retractor including a first retractor blade, a second retractor blade, at least one arm extending from said first retractor blade, and at least one arm extending from said second retractor blade; and
a cannula sized to slideably couple to said retractor;
wherein said cannula is configured to pivot along two axes when coupled to said retractor.

15. The less invasive access port of claim 14, wherein said arms are flexibly connected to said first and second retractor blade to allow said cannula to pivot in both an axial and a mediolateral direction.

16. The less invasive access port of claim 14, wherein:
said cannula further comprises a channel in a wall of said cannula fluidly connecting a top portion of said cannula to a bottom portion of said cannula; and
a housing coupled to said cannula, wherein said housing includes an interface port fluidly coupled to said cannula channels.

17. A method for accessing a surgical site using a less invasive access port, comprising:
inserting a k-wire into soft tissues around said surgical site;
percutaneously inserting at least one screw into said surgical site;
placing a trocar over the k-wire to dilate the soft tissues and provide access to the surgical site after said screw insertion;
inserting a retractor over said trocar down to said surgical site;
inserting a cannula into said retractor; and
opening said retractor to expose said surgical site.

18. The method of claim 17, further comprising removing said trocar.

19. The method of claim 17, further comprising inserting said screw, said k-wire, said trocar, and said retractor through a fascial plane lateral to a multifidous muscle.

20. The method of claim 17, further comprising inserting a fiber optic fiber in a first channel formed in a wall of said cannula to illuminate said surgical site.

21. The method of claim 20, further comprising aspirating or irrigating said surgical site through a second channel formed in a wall of said cannula.

22. The method of claim 17 further comprising locking said retractor in a deployed position.

23. A method for accessing a vertebrae comprising passing an instrument in a fascial plane lateral to a multifidous muscle.

24. A less invasive access portal device for use in minimally invasive surgery comprising:
a first expanding portion having a partial cylindrical shape;
a second expanding portion having a partial cylindrical shape;
at least one pivotal connection connecting the first expanding portion and the second expanding portion, wherein the first expanding portion and the second expanding portion form a relative cylinder when pivoted to a closed position;
at least one arm extending vertically upward from each of said expanding portions, wherein said arms provide leverage to open and close said expanding portion.

25. The less invasive access portal device of claim 24, further comprising a retaining member coupled to one of said arms, said retaining member being configured to releasably retain said expanding portions in a deployed position;
wherein said retaining member is configured to be outside a wound when said first and second expanding portions are deployed inside said wound.

26. A surgical retractor, comprising:
a first member having a first end and a second end, the first end extending at an angle with respect to the second end;
a second member having a first end and a second end;
the first member having two edges that face the second member and the second member having two edges that face the first member, the edges extending from the first end to the second end of the first and second members, the edges providing a lever abutment location to provide an axis for rotation of the first member and the second member with respect to each other to form a surgical retractor at the second end of the first and second members.

27. A less invasive access port, comprising:
a retractor; and
a cannula configured to be coupled to said retractor;
wherein said cannula includes a wall having a plurality of channels defined therein;
said channels fluidly connecting a top portion of said cannula to a bottom portion of said cannula.

28. The less invasive access port of claim 27, further comprising:
a housing fluidly connected to said cannula channels, said housing including at least one integrated interface port;
wherein said integrated interface port provides light, suction, and irrigation to the bottom portion of said cannula.
